(19) United States
(12) Patent Application Publication
(10) Pub. No.: US 2026/0130587 A1
(43) Pub. Date: May 14, 2026

(54) OPTICAL COMPONENT FOR OPHTHALMIC DIAGNOSTIC DEVICE

(71) Applicant: Alcon Inc., Fribourg (CH)

(72) Inventors: Christopher Paul Voita, Dove Canyon, CA (US); Jonathan Drewes, Oviedo, FL (US); Richard Ty Olmstead, Oviedo, FL (US)

(21) Appl. No.: 19/371,097

(22) Filed: Oct. 28, 2025

Related U.S. Application Data
(60) Provisional application No. 63/719,955, filed on Nov. 13, 2024.

(51) Int. Cl. A61B 3/10; A61B 3/14; G02B 3/00

(52) U.S. Cl. CPC A61B 3/1015; A61B 3/14; G02B 3/0056

(57) ABSTRACT

An optical component includes a substrate having a first optical surface located opposite a second optical surface with at least one of the first optical surface or the second optical surface including a lenslet array formed therein. The optical component also includes an anti-reflective coating located on the first optical surface, a bandpass filter coating located on the second optical surface, and at least one obscuration located on the substrate. The obscuration is configured to block a transmission of light between the first optical surface and the second optical surface.

### INTRODUCTION

The present disclosure relates to systems and methods utilizing an optical component in connection with a wavefront analyzer.

Such systems are commonly used during a refractive ophthalmic surgery, i.e. in surgical operations in which the cornea of a patient's eye is shaped by a laser beam in order to correct for defects of vision. Before the surgical operation, a measurement of the patient's eye is made with the patient usually sitting in an upright position while focusing on a target image. A so-called wavefront analyzer then objectively determines an appropriate wavefront correction for reshaping the cornea of the eye. Typically, the wavefront analyzer calculates a cylindrical or quasi-cylindrical ablation profile, which is applied to the eye by means of a focused laser beam.

### SUMMARY

Disclosed herein is an optical component. The component includes a substrate having a first optical surface located opposite a second optical surface with at least one of the first optical surface or the second optical surface including a lenslet array formed therein. The optical component also includes an anti-reflective coating located on the first optical surface, a bandpass filter coating located on the second optical surface, and at least one obscuration located on the substrate. The obscuration is configured to block a transmission of light between the first optical surface and the second optical surface.

In one aspect of the disclosure the substrate includes one of a circular edge geometry or a rectangular edge geometry.

In one aspect of the disclosure the substrate is configured to allow the transmission of light in at least one of an ultraviolet waveband, a visible waveband, or an infrared waveband.

In one aspect of the disclosure the lenslet array includes a plurality of lenslets each having one of a square, rectangular, circular, or hexagonal perimeter cross-sectional area.

In one aspect of the disclosure the lenslet array is formed in the first optical surface and the second optical surface is a planar surface.

In one aspect of the disclosure the at least one obscuration includes a first obscuration located on the first optical surface following a curvature of at least one lenslet of the lenslet array and a second obscuration located on the second optical surface with the first obscuration aligned relative to the second obscuration and the second obscuration being planar.

In one aspect of the disclosure the lenslet array includes a first lenslet array formed into the first optical surface and a second lenslet array formed into the second optical surface.

In one aspect of the disclosure each lenslet in the first lenslet array include a first curvature and each lenslet in the second lenslet array include a second curvature that can be equal to or different from the first curvature.

In one aspect of the disclosure the at least one obscuration includes a first obscuration and a second obscuration, the first obscuration follows the first curvature of at least one lenslet of the first lenslet array and the second obscuration follows the second curvature of at least one lenslet of the second lenslet array.

In one aspect of the disclosure a first pitch of each lenslet in the first lenslet array matches a second pitch of each lenslet in the second lenslet array.

In one aspect of the disclosure a first pitch of each lenslet in the first lenslet array is not equally spaced and a second pitch of each lenslet in the second lenslet array is not equally spaced but matches that of the first lenslet array and is aligned in an X-direction, a Y-direction, and a clocking angle.

Disclosed herein is an ophthalmic diagnostic device. The device includes a housing and a wavefront sensor located within the housing and configured to receive light from an optical component. The component includes a substrate having a first optical surface located opposite a second optical surface with at least one of the first optical surface or the second optical surface including a lenslet array formed therein. The optical component also includes an anti-reflective coating located on the first optical surface, a bandpass filter coating located on the second optical surface, and at least one obscuration located on the substrate. The obscuration is configured to block a transmission of light between the first optical surface and the second optical surface.

In one aspect of the disclosure the at least one obscuration includes a first obscuration located on the first optical surface following a curvature of at least one lenslet of the lenslet array and a second obscuration located on the second optical surface with the first obscuration aligned relative to the second obscuration and the second obscuration being planar.

In one aspect of the disclosure the first optical surface includes a first lenslet array formed therein and the second optical surface includes a second lenslet array formed therein.

In one aspect of the disclosure each lenslet in the first lenslet array includes a first curvature and each lenslet in the second lenslet array includes a second curvature can be equal to or different from the first curvature.

In one aspect of the disclosure the at least one obscuration includes a first obscuration and a second obscuration, the first obscuration follows the first curvature of at least one lenslet of the first lenslet array and the second obscuration follows the second curvature of at least one lenslet of the second lenslet array.

Disclosed herein is a method of forming an optical component. The method includes determining a lenslet configuration for the optical component. The lenslet configuration includes a lenslet array on at least one of a first optical surface on a substrate or a second optical surface on the substrate. The method also includes forming the lenslet configuration into at least one of the first optical surface or the second optical surface on the substrate and applying an anti-reflective coating to one of the first optical surface or the second optical surface and applying a bandpass filter coating to the other of the first optical surface or the second optical surface. The method also includes locating at least one obscuration on the substrate. The obscuration is configured to block the transmission of light through the substrate.

In one aspect of the disclosure locating the at least one obscuration on the substrate includes applying a first obscuration on the first optical surface and applying a second obscuration on the second optical surface that is aligned with the first obscuration.

In one aspect of the disclosure locating the at least one obscuration on the substrate includes positioning a single obscuration with an opening in the substrate that extends between the first optical surface and the second optical surface.

In one aspect of the disclosure the lenslet configuration includes a first lenslet array for the first optical surface and a second lenslet array for the second optical surface.

### BRIEF DESCRIPTION OF THE DRAWINGS

FIG. 1 is a schematic illustration of an exemplary surgical suite configured with a visualization system as set forth in detail herein.

FIG. 2 is a schematic illustration of an example optical component.

FIG. 3 is a cross-sectional view taken along line 3-3 of FIG. 2.

FIG. 4 is a cross-sectional view of another example optical component.

FIG. 5 is a cross-sectional view of yet another example optical component.

FIG. 6 is a cross-sectional view of a further example optical component.

FIG. 7 is a cross-sectional view of an example optical component having a first lenslet array located on a first optical surface and a second lenslet array located on a second optical surface.

FIG. 8 is a cross-sectional view of another example optical component having a first lenslet array located on a first optical surface and a second lenslet array located on a second optical surface.

FIG. 9 is a cross-sectional view of yet another example optical component having a first lenslet array located on a first optical surface and a second lenslet array located on a second optical surface.

FIG. 10 is a cross-sectional view of yet another example optical component having a first lenslet array located on a first optical surface and a second lenslet array located on a second optical surface.

FIG. 11 is a schematic illustration of another optical component.

FIG. 12 is a schematic illustration of yet another optical component.

FIG. 13 is a schematic illustration of a further example optical component.

FIG. 14 is a schematic illustration of a further example optical component.

FIG. 15 is a schematic illustration of a further example optical component.

FIG. 16 is a schematic illustration of a further example optical component.

FIG. 17 is a schematic illustration of a further example optical component.

FIG. 18 is a flowchart of an example method of forming an optical component.

The foregoing and other features of the present disclosure are more fully apparent from the following description and appended claims, taken in conjunction with the accompanying drawings.

### DETAILED DESCRIPTION

Embodiments of the present disclosure are described herein. It is to be understood, however, that the disclosed embodiments are merely examples and other embodiments can take various and alternative forms. The figures are not necessarily scaled. Some features could be exaggerated or minimized to show details of particular components. Therefore, specific structural and functional details disclosed herein are not to be interpreted as limiting, but merely as a representative basis for teaching one skilled in the art to variously employ the present disclosure. As those of ordinary skill in the art will understand, various features illustrated and described with reference to any one of the figures can be combined with features illustrated in one or more other figures to produce embodiments that are not explicitly illustrated or described. The combinations of features illustrated provide representative embodiments for typical applications. Various combinations and modifications of the features consistent with the teachings of this disclosure, however, could be desired for particular applications or implementations.

Certain terminology may be used in the following description for the purpose of reference only, and thus are not intended to be limiting. For example, terms such as “above” and “below” refer to directions in the drawings to which reference is made. Terms such as “front,” “back,” “fore,” “aft,” “left,” “right,” “rear,” and “side” describe the orientation and/or location of portions of the components or elements within a consistent but arbitrary frame of reference which is made clear by reference to the text and the associated drawings describing the components or elements under discussion. Moreover, terms such as “first,” “second,” “third,” and so on may be used to describe separate components. Such terminology may include the words specifically mentioned above, derivatives thereof, and words of similar import.

The disclosure is directed to an optical component for an ophthalmic diagnostic device. In one example, the diagnostic device obtains preoperative or postoperative measurements of a patient's eye. To obtain non-image preoperative or postoperative diagnostic information of the eye, the diagnostic device can utilize a Shack Hartmann style wavefront analyzer. Alternatively, the preoperative or postoperative diagnostic information can include image captures for measuring anatomical characteristics of the eye, such as with an optical coherence tomography design, to aid in calculating an appropriately powered intraocular lens (“IOL”). In another example, the diagnostic device can include an Optiwave Refractive Analysis (“ORA”) System® device capable of obtaining intraoperative measurements through a non-imaging wavefront analyzer, such as one utilizing a Talbot-Moire design.

In one example, the optical component used in connection with the above identified example diagnostic devices includes a substrate having a first optical surface and a second optical surface opposite the first optical surface. At least one of the first or second optical surfaces include a lenslet array formed therein. The lenslet array divides an incoming wavefront into sub-apertures for wavefront characterization. Additionally, the optical component includes one of an anti-reflective coating located on one of the first or the second optical surfaces and a narrow bandpass filter coating located on the other of the first or the second optical surfaces. The narrow bandpass filter coating is configured to pass only the wavelength(s) of a sensor on the diagnostic device to enhance the signal-to-noise ratio to improve accuracy of the data captured. The optical component also includes an obscuration that minimizes or eliminates axial specular reflections from reaching the sensor.

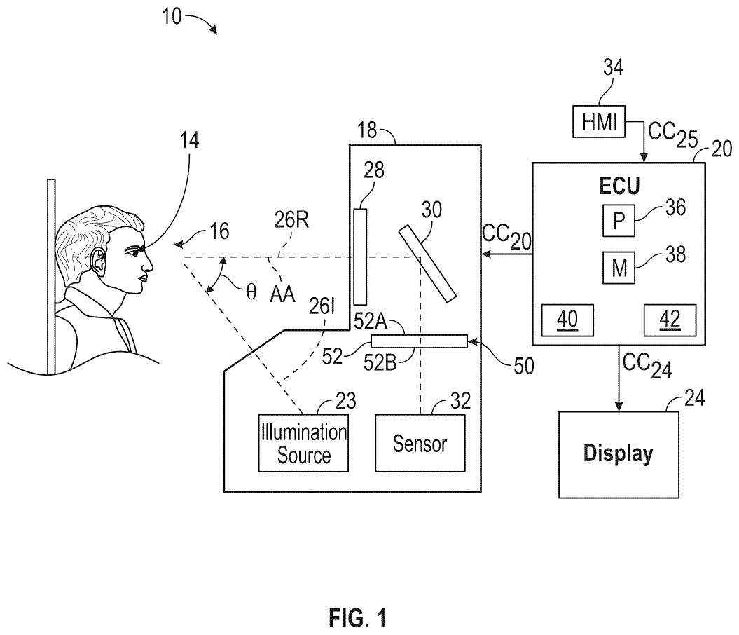

Referring to the drawings, wherein like reference numbers refer to like components, a representative diagnostic device 10 is depicted schematically in FIG. 1. The diagnostic device 10 obtains measurements of a target eye 14 of a patient 16.

As contemplated herein, representative ophthalmic procedures performable in connection with the diagnostic device 10 of FIG. 1 include lens replacement surgeries, e.g., cataract surgeries, or refractive lens exchanges (RLEs).

An electronic control unit (ECU) 20 is also present within the diagnostic device 10 of FIG. 1. The ECU 20, which within the scope of the disclosure is used with or as an integral part of the diagnostic device 10, is programmed in software and equipped in hardware, i.e., configured, to execute computer readable instructions to generate diagnostic information for the target eye 14. For example, the diagnostic information obtained by the diagnostic device 10 is sent through control signals CC₂₄ to a display 24 for viewing by the user.

Although the ECU 20 shown in FIG. 1 is depicted as a unitary box for illustrative clarity and simplicity, the ECU 20 within the scope of the disclosure could include one or more networked devices each with a central processing unit or other processor (P) 36 and sufficient amounts of memory (M) 38, including a non-transitory (e.g., tangible) storage medium that participates in providing data/instructions that may be read by the processor(s) 36. Instructions embodying diagnostic information collection 40 may be stored in the memory 38 and executed by the processor 36 to perform the various functions described herein.

The memory 38 may take many forms, including but not limited to non-volatile media and volatile media. Non-volatile media may include optical and/or magnetic disks or other persistent memory, while volatile media may include dynamic random-access memory (DRAM), static RAM (SRAM), etc., any or all which may constitute a main memory of the ECU 20. Input/output (I/O) circuitry 42 may be used to facilitate connection to and communication with the various peripheral devices used during the ophthalmic procedure, inclusive of the various hardware of the diagnostic device 10 of FIG. 1.

Other hardware not depicted but commonly used in the art may be included as part of the ECU 20, including but not limited to a local oscillator or high-speed clock, signal buffers, filters, etc. A human machine interface (HMI) 34 may be included within the structure of the diagnostic device 10 to allow the user to interact with the ECU 20, e.g., via input signals (arrow CC₂₅). The ECU 20 may also control the diagnostic device 10 directly, e.g., via control signals (arrow CC₂₀), or via the input signals (arrow CC₂₅) in different embodiments. Various implementations of the HMI 34 may be used within the scope of the present disclosure, including but not limited to a footswitch, a touch screen, buttons, control knobs, a speaker for voice activation, etc. The ECU 20 of FIG. 1 may be configured to communicate via a network (not shown), for instance a serial bus, a local area network, a controller area network, a controller area network with flexible data rate, or via Ethernet, Wi-Fi, Bluetooth™, near-field communication, and/or other forms of wired or wireless data connection.

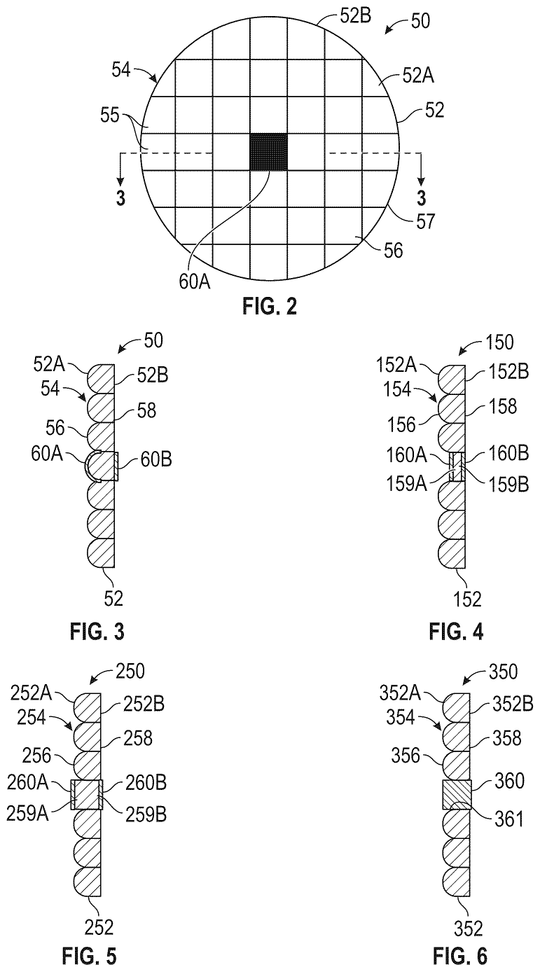

With reference to FIG. 2 and continued reference to FIG. 1, the example diagnostic device 10 can include a housing 18 for enclosing various components. In the illustrated example, the housing 18 encloses an illumination source 23 for generating input light beams 26I that reflect off of the target eye 14 at angle θ to generate reflected light beams 26R that are collected by the diagnostic device 10. The reflected light beams 26R travel along an optical axis AA to a polarizer 28. From the polarizer 28, the reflected light beams 26R reflect off a mirror 30 and into an optical component 50. From the optical component, the reflected light beams 26R reach a sensor 32.

In the illustrated example, the optical component 50 includes a substrate 52 having a first optical surface 52A and a second optical surface 52B opposite the first optical surface 52A. The substrate 52 is comprised of a material that allows for the transmission of light in at least one of the ultraviolet, visible, or infrared wavebands between the first and second optical surfaces 52A, 52B.

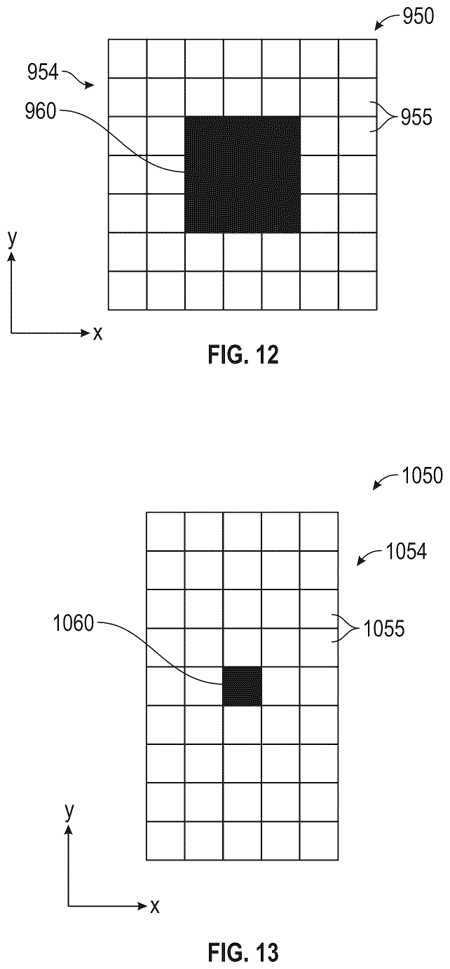

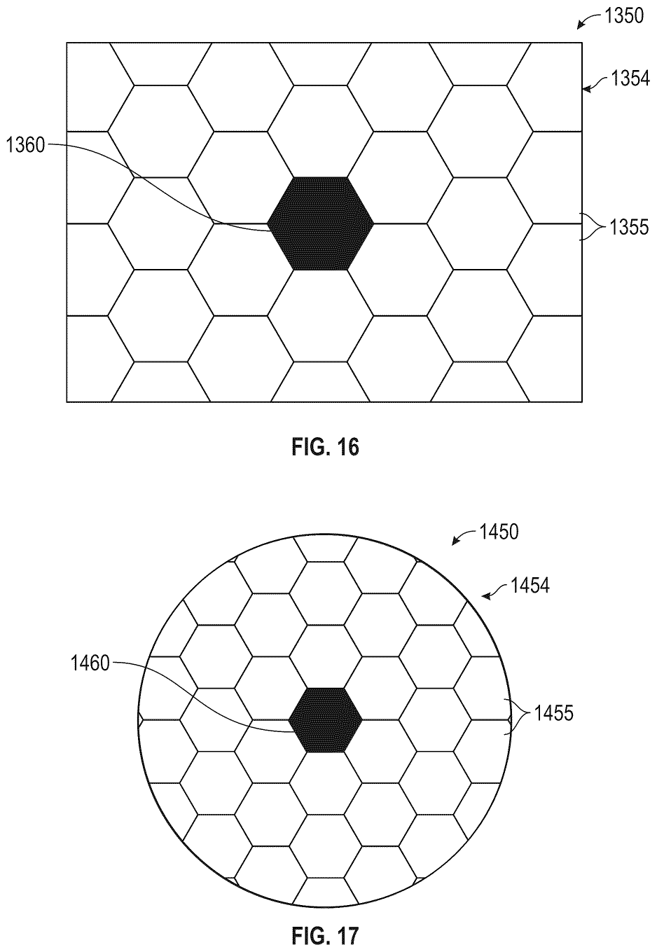

In the illustrated example, a lenslet array 54 having lenslets 55 is formed into at least one of the first optical surface 52A or the second optical surface 52B in a grid pattern. An edge 57 of the substrate 52 defines a circular perimeter of the substrate 52, however, an edge of the substrate 52 can embody other geometric shapes, such as rectangular (FIG. 13) or square (FIG. 12) as discussed further below. The lenslet array 54 can be formed into the substrate 52 through a variety processes, such as but not limited to molding, casting, hot embossing, inkjet printing, laser based, or a photolithographic technique. The lenslet array 54 may be refractive or diffractive, have a number of different base surface profiles (e.g., plano, convex, concave, spherical, aspherical, etc.), have varying pitch or spacing, varying base radius of curvature, different perimeter edge shapes, or can be arranged in any geometry (e.g., square pack FIG. 2, hexagonal pack (FIGS. 16-17, etc.).

As shown in FIGS. 2-3, the first optical surface 52A includes an anti-reflective coating 56. One feature of the anti-reflective coating 56 is to enhance transmission of the wavefront sensor system wavelength(s). The second optical surface 52B includes a filter coating 58, such as a narrow bypass filter coating, located thereon. In one example, the anti-reflective coating 56 and the filter coating 58 are located over the entirety of the first optical surface 52A and the second optical surface 52B, respectively. In another example, the anti-reflective coating 56 and the filter coating 58 are located in a predetermined pattern or on selected areas of the first optical surface 52A and the second optical surface 52B, respectively. Alternatively, the filter coating 58 can be located on the first optical surface 52A and the anti-reflective coating 56 can be located on the second optical surface 52B in the configurations mentioned above.

As shown in FIGS. 2-3, a first obscuration 60A is located on the first optical surface 52A and a second obscuration 60B is located on the second optical surface 52B. The first and second obscurations 60A, 60B stop the transmission of light through the substrate 52. In one example, the first and second obscurations 60A, 60B include a dimension equal to a single lenslet (FIGS. 2-3 and 11) of the lenslet array 54. In another example, the first and second obscurations 60A, 60B include a dimension equal to multiple lenslets of the lenslet array 54 (FIG. 12).

As shown in FIGS. 2-3, the individual lenslets in the lenslet array 54 have refractive convex surface profiles with the lenslets being square and in a square pack geometry that are covered in the anti-reflective coating 56. The second optical surface 52B is planar and is coated with the filter coating 58. The first obscuration 60A follows a curvature of one of the lenslets of the lenslet array 54 and the second obscuration 60B that is located on the second optical surface 52B is planar. The first and second obscurations 60A, 60B are aligned with each other in position and clocking angle along a center of the substrate 52 and their locations with respect to the edge of the substrate 52 are also accurately positioned.

FIG. 4 illustrates another example optical component 150. The optical component 150 is similar to the optical component 50 except where described below or shown in the drawings. Like or similar components will include the addition of a leading “1.”

In the illustrated example, the optical component 150 includes a substrate 152 having a lenslet array 154 formed in a first optical surface 152A with a second optical surface 152B located opposite the first optical surface 152A. An anti-reflective coating 156 is located on the first optical surface 152A and a filter coating 158 is located on the second optical surface 152B.

A first obscuration 160A is located on a first planar surface 159A of the first optical surface 152A and a second obscuration 160B is located on a second planar surface 159B of the second optical surface 152B. The first and second obscurations 160A, 160B are both planar and an individual grid of the lenslet array 154 that would have included a lenslet is formed as a planar surface in the substrate 152 to accommodate the planar profile of the first obscuration 160A. In particular, the planar surface 159A of the substrate 152 that accommodates the first obscuration 160A is aligned with a base portion of adjacent lenslets in the lenslet array 154.

FIG. 5 illustrates another example optical component 250. The optical component 250 is similar to the optical component 50 except where described below or shown in the drawings. Like or similar components will include the addition of a leading “2.”

In the illustrated example, the optical component 250 includes a substrate 252 having a lenslet array 254 formed in a first optical surface 252A with a second optical surface 252B opposite the first optical surface 252A. An anti-reflective coating 256 is located on the first optical surface 252A and a filter coating 258 is located on the second optical surface 252B.

A first obscuration 260A is located on a first planar surface 259A of the first optical surface 252A and a second obscuration 260B is located on a second planar surface 259B of the second optical surface 252B. The first and second obscurations 260A, 260B are both planar and an individual grid of the lenslet array 254 that would have included a lenslet is formed as a planar surface in the substrate 252 to accommodate the planar profile the first obscuration 260A. In particular, the planar surface 259A of the substrate 252 that accommodates the first obscuration 260A is aligned with a peak of adjacent lenslets of the lenslet array 254 or with an original thickness of the substrate 252. The original thickness of the substrate 252 can correspond to a surface of the substrate 252 that was not changed during the formation of the adjacent lenslets of the lenslet array 254.

FIG. 6 illustrates another example optical component 350. The optical component 350 is similar to the optical component 50 except where described below or shown in the drawings. Like or similar components will include the addition of a leading “3.”

In the illustrated example, the optical component 350 includes a substrate 352 having a lenslet array 354 formed into a first optical surface 352A with a second optical surface 352B opposite the first optical surface 352A. An anti-reflective coating 356 is located on the first optical surface 352A and a filter coating 358 is located on the second optical surface 352B.

A single obscuration 360 is located in an opening 361 in the substrate 352 and extends through the substrate 352 between the first optical surface 352A and the second optical surface 352B. In the illustrated example, the obscuration 360 is cubic in shape and corresponds to a size of a single lenslet of the lenslet array 354 that has been removed from the substrate 352 to accommodate the obscuration 360 therein.

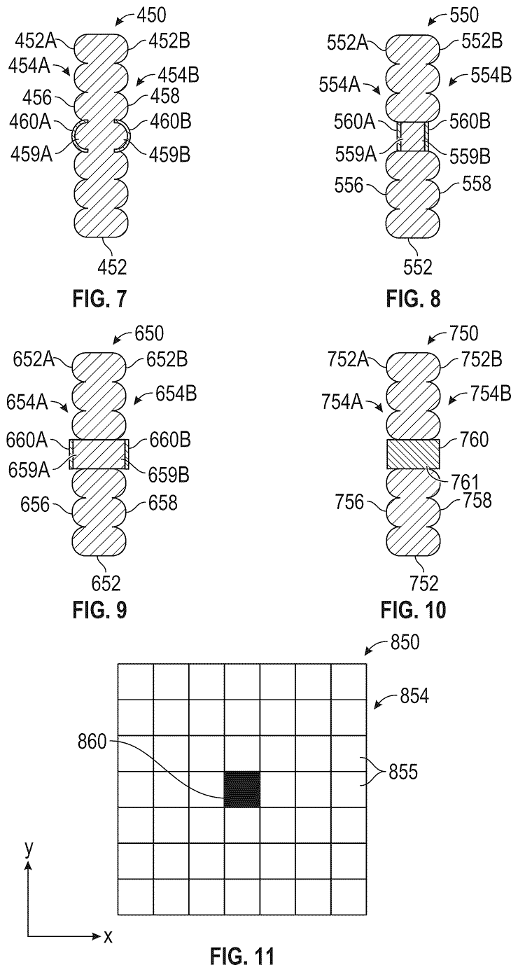

FIG. 7 illustrates another example optical component 450. The optical component 450 is similar to the optical component 50 except where described below or shown in the drawings. Like or similar components will include the addition of a leading “4.”

In the illustrated example, the optical component 450 includes a substrate 452 having a first optical surface 452A located opposite a second optical surface 452B. The substrate 452 includes a first lenslet array 454A formed in a grid pattern into the first optical surface 452A and a second lenslet array 454B formed in a grid pattern into the second optical surface 452B. The individual lenslets in the first and second lenslet arrays 454A, 454B have refractive convex surface profiles with the lenslets being in a square pack geometry.

A grid pattern of the first lenslet array 454A is aligned with a grid pattern of the second optical array 454B. However, the first and second lenslet arrays 454A and 454B may be refractive or diffractive, have a number of different base surface profile (e.g., plano, convex, concave, spherical, aspherical, etc.), have varying pitch or spacing, varying base radius of curvature, different perimeter edge shapes, or can be arranged in any geometry (e.g., square pack, hexagonal pack, etc.).

In the illustrated example, an anti-reflective coating 456 is located on the first optical surface 452A and a filter coating 458, such as a narrow bypass filter coating, is located on the second optical surface 452B. One feature of the anti-reflective coating 456 is to enhance transmission of the wavefront sensor system wavelength(s).

In one example, the anti-reflective coating 456 and the filter coating 458 can be located over the entire first and second optical surfaces 452A, 452B, respectively. In another example, the anti-reflective coating 456 and the filter coating 458 can be located over predetermined or selected areas first and second optical surfaces 452A, 452B, respectively. Furthermore, the filter coating 458 can be located on the first optical surface 452A and the anti-reflective coating 456 can be located on the second optical surface 452B.

A first obscuration 460A is located on the first optical surface 452A and follows a profile of one of the lenslets in the first lenslet array 454A and a second obscuration 460B is located on the second optical surface 452B and follows a profile of one of the lenslets in the second lenslet array 454B. The first and second obscurations 460A, 460B stop the transmission of light through the substrate 452. In one example, the first and second obscurations 460A, 460B include a dimension equal to a single lenslet of the first and second lenslet arrays 454A, 454B respectively. In another example, the first and second obscurations 460A, 460B include a dimension equal to multiple lenslets of the first and second lenslet arrays 454A, 454B, respectively. The first obscuration 460A is located on a first curved surface 459A of the first optical surface 452A and the second obscuration 460B is located on a second curved surface 459B of the second optical surface 452B. The first and second obscurations 460A, 460B are aligned in position and clocking angle along the center of the substrate 452 and their locations with respect to the edge of the substrate 452.

FIG. 8 illustrates another example optical component 550. The optical component 550 is similar to the optical components 50 and 450 except where described below or shown in the drawings. Like or similar components will include the addition of a leading “5.”

In the illustrated example, the optical component 550 includes a substrate 552 having a first lenslet array 554A formed into a first optical surface 552A and a second lenslet array 554B formed into a second optical surface 552B opposite the first optical surface 552A. The first optical surface 552A includes an anti-reflective coating 556 and the second optical surface 552B includes a filter coating 558.

A first obscuration 560A is located on a first planar surface 559A of the first optical surface 552A and a second obscuration 560B is located on a second planar surface 559B of the second optical surface 552B. The first and second obscurations 560A, 560B are both planar and located in place of corresponding lenslets in the first and second lenslet arrays 554A, 554B, respectively. In particular, the first and second planar surfaces 559A, 559B of the substrate 552 are aligned with base portions of the adjacent lenslets in the first and second lenslet arrays 554A, 554B.

FIG. 9 illustrates another example optical component 650. The optical component 650 is similar to the optical components 50 and 450 except where described below or shown in the drawings. Like or similar components will include the addition of a leading “6.”

In the illustrated example, the optical component 650 includes a substrate 652 having a first lenslet array 654A formed into a first optical surface 652A and a second lenslet array 654B formed into a second optical surface 652B opposite the first optical surface 652A. The first optical surface 652A includes an anti-reflective coating 656 and the second optical surface 652B includes a filter coating 658.

A first obscuration 660A is located on a first planar surface 659A of the first optical surface 652A and a second obscuration 660B is located on a second planar surface 659B of the second optical surface 654B. The first and second obscurations 660A, 660B are both planar and located in place of corresponding lenslets in the first and second lenslet arrays 654A, 654B, respectively. In one example, the first and second planar surfaces 659A, 659B are aligned with an original surface of the substrate 652 prior to forming the first and second lenslet arrays 654A, 654B. In another example, the first and second planar surfaces 659A, 659B are aligned with a peak of each of the lenslets in the first and second lenslet arrays 654A, 654B.

FIG. 10 illustrates another example optical component 750. The optical component 750 is similar to the optical components 50 and 450 except where described below or shown in the drawings. Like or similar components will include the addition of a leading “7.”

In the illustrated example, the optical component 750 includes a substrate 752 having a first lenslet array 754A formed into a first optical surface 752A and a second lenslet array 754B formed into a second optical surface 752B opposite the first optical surface 752A. The first optical surface 752A includes an anti-reflective coating 756 and the second optical surface 752B includes a filter coating 758.

An obscuration 760 is located within an opening 761 in the substrate 752 and extends through the substrate 752 between the first optical surface 752A and the second optical surface 752B. In the illustrated example, the obscuration 760 is cubic and corresponds to a size of a single lenslet in each of the first and second lenslet arrays 754A, 754B to accommodate the obscuration 760 therein.

FIG. 11 illustrates another example optical component 850 having a square grid pattern with an equal number of lenslets 855 in a lenslet array 854 extending in an X-direction as compared to a Y-direction. The optical component 850 can include any of the configurations described above in FIGS. 2-10. As shown in FIG. 11, an obscuration 860 is located in a center of the grid pattern with the grid pattern having an odd number of lenslets 855 in each dimension that form the lenslet array 854.

FIG. 12 illustrates another example optical component 950 having a square grid pattern with an equal number of lenslets 955 in a lenslet array 954 extending in an X-direction as compared to a Y-direction. The optical component 950 can include any of the configurations described above in FIGS. 2-10. As shown in FIG. 12, an obscuration 960 is located in a center of the grid pattern with the grid pattern having an odd total number of lenslets 955 in an X-direction and a Y-direction that form the lenslet array 954. In the illustrated example, the obscuration 960 encompasses an area of the grid pattern having more than one lenslet 955, such as nine square lenslets.

FIG. 13 illustrates another example optical component 1050 having a rectangular grid pattern with a greater number of lenslets 1055 in a lenslet array 1054 extending in a Y-direction compared to an X-direction. The optical component 1050 can include any of the configurations described above in FIGS. 2-10. As shown in FIG. 13, an obscuration 1060 is located in a center of the grid pattern with the grid pattern having an odd number of lenslets 1055 in the X-direction and the Y-direction that form a lenslet array 1054.

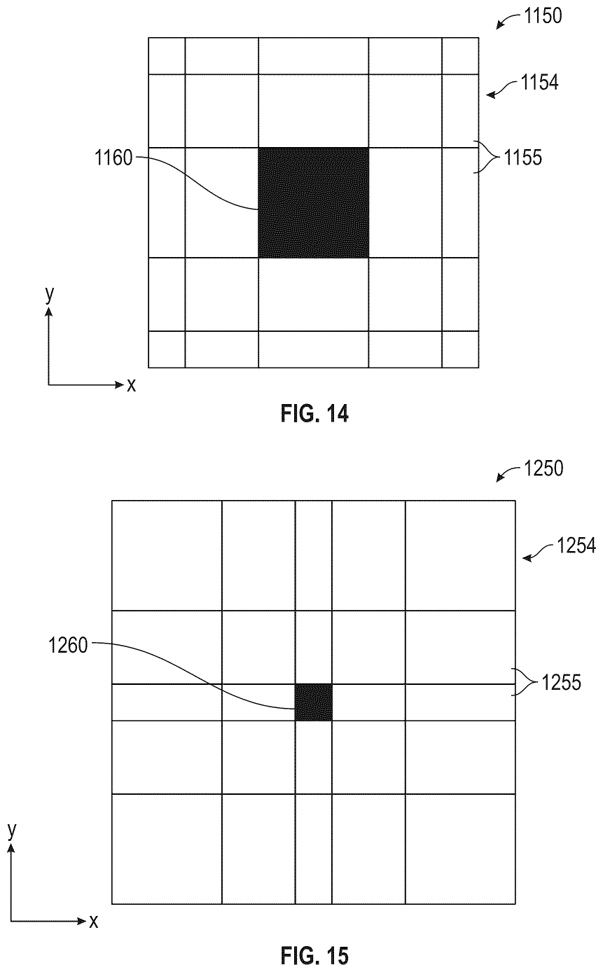

FIG. 14 illustrates another example optical component 1150 having a grid pattern with an equal number of lenslets 1155 in a lenslet array 1154 extending in an X-direction as compared to a Y-direction. The optical component 1150 can include any of the configurations described above in FIGS. 2-10. As shown in FIG. 14, an obscuration 1160 is located in a center of the grid pattern with the grid pattern having an odd number of lenslets 1155 in each dimension that form the lenslet array 1154. Furthermore, the lenslets 1155 in the lenslet array 1154 include a non-uniform geometry such that some of the lenslets 1155 may include varying dimensions in the X and Y-directions.

FIG. 15 illustrates another example optical component 1250 having a grid pattern with an equal number of lenslets 1255 in a lenslet array 1254 extending in an X-direction as compared to a Y-direction. The optical component 1250 can include any of the configurations described above in FIGS. 2-10. As shown in FIG. 15, an obscuration 1260 is located in a center of the grid pattern with the grid pattern having an odd number of lenslets 1255 in each dimension that form the lenslet array 1254. Furthermore, the lenslets 1255 in the lenslet array 1254 include a non-uniform geometry such that some of the lenslets 1255 may include varying dimensions in the X and Y-directions.

FIG. 16 illustrates another example optical component 1350 having a hexagonal pattern with an unequal number of lenslets 1355 in a lenslet array 1354 extending in an X-direction as compared to a Y-direction. The optical component 1350 can include any of the configurations described above in FIGS. 2-10. As shown in FIG. 15, an obscuration 1360 is located in a center of the component with the hexagonal pattern having an odd number of lenslets 1355 in the X-direction and an even number of lenslets 1355 in the Y-direction. The component 1350 also includes an edge that defines a rectangular profile.

FIG. 17 illustrates another example optical component 1450 having a hexagonal pattern having lenslets 1455 in a lenslet array 1454 that include a circular edge profile. The optical component 1450 can include any of the configurations described above in FIGS. 2-10. As shown in FIG. 17, an obscuration 1460 is located in a center of the component with the hexagonal pattern having a single hexagonal obscuration in the center, with n successive “rings” of lenslets 1455 surrounding the center obscuration with n times six lenslets 1455 in each ring starting at ring n=1, with the outermost ring containing partial lenslets 1455 with individual areas less than that of a single full lenslet 1455 due to the geometry of a circular perimeter circumscribed on a hexagonal array.

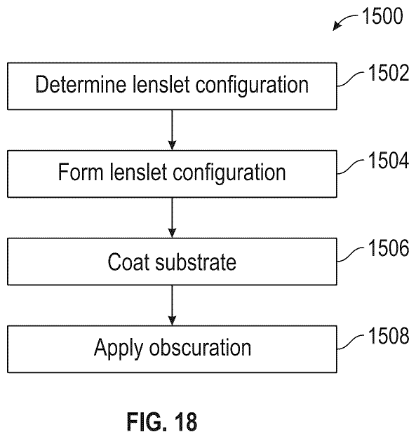

FIG. 18 illustrates a method 1500 of forming an optical component. The method 1500 begins at block 1502 (“Determine Lenslet Configuration”). At block 1502, a configuration for the lenslets that will form the lenslet array is determined for at least one of a first optical surface or a second optical surface of the substrate. As discussed above, the configuration for the lenslets can include refractive or diffractive, base surface profile (e.g., plano, convex, concave, spherical, aspherical, etc.), varying pitch or spacing, varying base radius of curvature, perimeter edge shapes, and arrangement of geometry (e.g., square pack, hexagonal pack, etc.). With the lenslet configuration determined, the method 1500 can then proceed to block 1504.

At block 1504 (“Form Lenslet Configuration”), the method 1500 forms the lenslet pattern into at least one of the first or second optical surfaces of the substrate. In one example, the grid pattern can be formed by a photolithographic or other technique capable of forming the lenslets in substrate. With the lenslet pattern formed into the substrate, the method 1500 proceeds to block 1506.

A block 1506 (“Coat Substrate”), the method applies a coating to a first optical surface and a second optical surface. One of the first and second optical surfaces can include an anti-reflective coating and the other of the first and second optical surfaces can include a bandpass filter coating. The coatings can be applied to the entire first and second optical surfaces on the substrate or in a predetermined pattern on less than the entire first and second optical surfaces. For example, the coatings can be applied to a predetermined number of lenslets that are less than a total number of lenslets in an array in either of the first and second surfaces. In another example, the predetermined number of lenslets includes lenslets from a first lenslet array on the first optical surface that align with lenslets from a second lenslet array on the second optical surface. With at least one coating applied, the method 1500 then proceeds to block 1508.

At block 1508 (“Apply Obscuration”), the method 1500 positions at least one obscuration relative to the substrate. In particular, the obscuration can include a first obscuration and a second obscuration as described above with respect to FIGS. 3-5 and 7-9 or a single obscuration that extends between the first and second optical surfaces of the substrate. With the obscuration applied to the substrate, the optical component can be completed and prepared for installation into diagnostic device 10 or another type of device.

Furthermore, the embodiments shown in the drawings, or the characteristics of various embodiments mentioned in the present description are not necessarily to be understood as embodiments independent of each other. Rather, it is possible that each of the characteristics described in one of the examples of an embodiment can be combined with one or a plurality of other desired characteristics from other embodiments, resulting in other embodiments not described in words or by reference to the drawings. Accordingly, such other embodiments fall within the framework of the scope of the appended claims.

1. An optical component, comprising:
a substrate having a first optical surface located opposite a second optical surface, wherein at least one of the first optical surface or the second optical surface includes a lenslet array formed therein;
an anti-reflective coating located on the first optical surface;
a bandpass filter coating located on the second optical surface; and
at least one obscuration located on the substrate and configured to block a transmission of light between the first optical surface and the second optical surface.

2. The optical component of claim 1, wherein the substrate includes one of a circular edge geometry or a rectangular edge geometry.

3. The optical component of claim 1, wherein the substrate is configured to allow the transmission of light in at least one of an ultraviolet waveband, a visible waveband, or an infrared waveband.

4. The optical component of claim 1, wherein the lenslet array includes a plurality of lenslets each having one of a square, rectangular, circular, or hexagonal perimeter cross-sectional area.

5. The optical component of claim 1, wherein the lenslet array is formed in the first optical surface and the second optical surface is a planar surface.

6. The optical component of claim 1, wherein the at least one obscuration includes a first obscuration located on the first optical surface following a curvature of at least one lenslet of the lenslet array and a second obscuration located on the second optical surface with the first obscuration aligned relative to the second obscuration and the second obscuration being planar.

7. The optical component of claim 1, wherein the lenslet array includes a first lenslet array formed into the first optical surface and a second lenslet array formed into the second optical surface.

8. The optical component of claim 7, wherein each lenslet in the first lenslet array include a first curvature and each lenslet in the second lenslet array include a second curvature that can be equal to or different from the first curvature.

9. The optical component of claim 8, wherein the at least one obscuration includes a first obscuration and a second obscuration, the first obscuration follows the first curvature of at least one lenslet of the first lenslet array and the second obscuration follows the second curvature of at least one lenslet of the second lenslet array.

10. The optical component of claim 8, wherein a first pitch of each lenslet in the first lenslet array matches a second pitch of each lenslet in the second lenslet array.

11. The optical component of claim 7, wherein a first pitch of each lenslet in the first lenslet array is not equally spaced and a second pitch of each lenslet in the second lenslet array is not equally spaced but matches that of the first lenslet array and is aligned in an X-direction, a Y-direction, and a clocking angle.

12. An ophthalmic diagnostic device, comprising:
a housing;
a wavefront sensor located within the housing and configured to receive light from an optical component, wherein the optical component includes:
a substrate having a first optical surface located opposite a second optical surface, wherein at least one of the first optical surface or the second optical surface includes a lenslet array formed therein;
an anti-reflective coating located on the first optical surface;
a bandpass filter coating located to the second optical surface; and
at least one obscuration located on the substrate and configured to block a transmission of light between the first optical surface and the second optical surface.

13. The ophthalmic diagnostic device of claim 12, wherein the at least one obscuration includes a first obscuration located on the first optical surface following a curvature of at least one lenslet of the lenslet array and a second obscuration located on the second optical surface with the first obscuration aligned relative to the second obscuration and the second obscuration being planar.

14. The ophthalmic diagnostic device of claim 12, wherein the first optical surface includes a first lenslet array formed therein and the second optical surface includes a second lenslet array formed therein.

15. The ophthalmic diagnostic device of claim 14, wherein each lenslet in the first lenslet array include a first curvature and each lenslet in the second lenslet array include a second curvature can be equal to or different from the first curvature.

16. The ophthalmic diagnostic device of claim 15, wherein that at least one obscuration includes a first obscuration and a second obscuration, the first obscuration follows the first curvature of at least one lenslet of the first lenslet array and the second obscuration follows the second curvature of at least one lenslet of the second lenslet array.

17. A method of forming an optical component, the method comprising:
determining a lenslet configuration for the optical component, wherein the lenslet configuration includes a lenslet array on at least one of a first optical surface on a substrate or a second optical surface on the substrate;
forming the lenslet configuration into at least one of the first optical surface or the second optical surface on the substrate;
applying an anti-reflective coating to one of the first optical surface or the second optical surface and applying a bandpass filter coating to the other of the first optical surface or the second optical surface; and
locating at least one obscuration on the substrate, wherein the obscuration is configured to block the transmission of light through the substrate.

18. The method of claim 17, wherein locating the at least one obscuration on the substrate includes applying a first obscuration on the first optical surface and applying a second obscuration on the second optical surface that is aligned with the first obscuration.

19. The method of claim 17, wherein locating the at least one obscuration on the substrate includes positioning a single obscuration with an opening in the substrate that extends between the first optical surface and the second optical surface.

20. The method of claim 17, wherein the lenslet configuration includes a first lenslet array for the first optical surface and a second lenslet array for the second optical surface.
